(19) United States
(12) Patent Application Publication
(10) Pub. No.: US 2016/0296270 A1
(43) Pub. Date: Oct. 13, 2016

(54) DEVICES AND METHODS FOR PROVIDING ADDITIONAL POWER TO SURGICAL DEVICES

(71) Applicant: Ethicon Endo-Surgery, LLC, Guaynabo, PR (US)

(72) Inventors: Geoffrey S. Strobl, Williamsburg, OH (US); Mark A. Davison, Mason, OH (US); Carl J. Draginoff, Jr., Mason, OH (US); Patrick M. Schleitweiler, West Chester, OH (US); Eric Johnson, Maineville, OH (US); Jason R. Lesko, Harrison, OH (US)

(21) Appl. No.: 14/683,255

(22) Filed: Apr. 10, 2015

(51) Int. Cl. A61B 18/12; A61N 7/00

(52) U.S. Cl. CPC A61B 18/1206; A61N 7/00; A61N 2007/0073; A61B 2018/128

(57) ABSTRACT

Devices and methods are described herein for providing enhanced power to a surgical device from a secondary power supply that operates in parallel to a primary power supply that provides power for therapeutic functions of the device. The secondary power supply can provide additional power for the therapeutic functions of the device, and/or it can provide power for non-therapeutic functions of the device such as sensors, displays, motors, etc. Subsystems powered by the primary power supply can be wholly isolated from subsystems powered by the secondary power supply, thus helping to prevent faults in one subsystem from affecting the other and providing secondary power without the need to modify the subsystems powered by the primary power supply. According to any of the systems described herein, additional power can thus be supplied to the device without affecting the structure and/or function of existing subsystems configured to supply primary power.

### FIELD

The present invention relates to devices and methods for use in surgical procedures and, more particularly, to providing additional power from one or more secondary power supplies for powered surgical devices and methods.

### BACKGROUND

Surgical devices for applying energy to tissue in order to treat and/or destroy the tissue are commonly used in surgical procedures. Such devices may comprise a hand piece and an instrument having a distally-mounted end effector. The end effector can be positioned against the tissue such that energy, commonly in the form of radio frequency (“RF”) or ultrasonic energy, is introduced into the tissue to provide, for example, substantially simultaneous transection of tissue and homeostasis by coagulation that can minimize patient trauma.

Energy applied by a surgical device can be transmitted to the device by a generator in communication with a hand piece of the device. For example, in the exemplary embodiment illustrated in FIG. 1, a generator 100 plugged into an alternating current (AC) power source 10 is configured to supply energy to a surgical device 200 via a cable plug 210. The cable plug 210 transmits power to a therapeutic energy circuit 214, e.g., to activate an end effector 250 of the device 200 to treat tissue. However, the generator 100 is traditionally configured to supply only enough power for the end effector 250 to operate on tissue, thereby limiting the functionality of the device 200 to therapeutic functions of the end effector 250. For example, in generator-connected surgical devices it is not currently feasible to power components that consume high levels of power and/or that require a different type of power than the end effector such as, for example, motors, powered sensors, microprocessors, etc.

Entirely replacing the generator 100 with a new, enhanced power supply device would be inefficient and expensive, as most operating rooms are already equipped with generators that are specifically configured for use with particular devices. Accordingly, there is a need in the art for improved devices and methods that provide enhanced power for additional functionalities of surgical devices without affecting the architecture of the primary power supply.

### SUMMARY

Devices and methods are generally provided for supplying enhanced power to a surgical instrument. In one exemplary embodiment, a system for supplying enhanced power to a surgical instrument includes a generator having a first power supply that is configured to supply power to a surgical instrument as part of a first power subsystem and a pass through power tap adapter configured to electrically couple to a second power supply as part of a second power subsystem. The second power subsystem supplies additional power to the surgical instrument that receives power from the generator, and the pass through power tap adapter is configured to isolate the second power subsystem from the first power subsystem.

In some embodiments, the power supplied by the first power supply can include alternating current and the power supplied by the second power supply can include direct current. The first power subsystem can be configured to supply power to a therapeutic end effector of a surgical instrument that receives power from the first and second power subsystems, and the second power subsystem can be configured to supply power to a non-therapeutic feature of the surgical instrument that receives power from the first and second power subsystems. In other embodiments, both the first and second power subsystems can be configured to supply power to a therapeutic end effector of the surgical instrument. The system can further include a means for isolated communication between the first and second subsystem. The means can be, by way of non-limiting examples, an opto-isolator, an inductive current sensor, or an RF transmitter and receiver.

The pass through power tap adapter can include a connector and an alternating current adapter. The connector can have a first end that is configured to couple to the generator, a second end that is configured to couple to the surgical instrument, and a pass-through outlet. The alternating current adapter can have a first end configured to electrically couple with the pass-through outlet and a second end that is configured to electrically couple to the second power supply such that power from the second power supply can be supplied through the alternating current adapter, through the connector, and to the surgical instrument. The connector can be removably and replaceably coupled to the generator. In alternative embodiments the connector can be fixedly coupled to the generator such that the connector cannot readily be decoupled from the generator.

The system can include a surgical instrument. The surgical instrument can be electrically coupled to each of the first and second power subsystems. In some embodiments the surgical instrument can be motor-driven. The motor may be used, by way of non-limiting examples, to open and close the end effector, advance and retract the knife, or to articulate and straighten the end effector. The surgical instrument can also have an actuable I-blade to transect tissue in some embodiments. In other embodiments the surgical instrument can contain lights for enhancing visualization or sensors for controlling power delivery to tissue.

In one exemplary embodiment of a system for supplying enhanced power during surgery, the system includes a generator, a surgical instrument adapter, and a cable. The generator includes a housing having a primary power supply and a secondary power supply disposed in it. The housing includes a front panel that is configured to allow a first port of the generator to be accessible to receive a surgical instrument adapter, with the first port providing access to the primary power supply. The housing also includes a back panel or a side panel that is configured to allow a second port of the generator to be accessible to provide access to the secondary power supply. The surgical instrument adapter is disposed in the first port such that a surgical instrument can be coupled to the adapter to electrically couple the surgical instrument to the primary power supply. The cable is coupled at a first end to the second port to electrically couple the cable to the secondary power supply. This allows the power from the secondary power supply to be supplied to an object coupled to a second end of the cable.

The secondary power supply of the system can be configured to provide power to an object that is coupled to the second end of the cable when power is not being provided to a surgical instrument that is electrically coupled to the first power supply. In some embodiments, power provided by the first power supply can be configured to power a therapeutic end effector of a surgical instrument and power provided by the second power supply can be configured to power a non-therapeutic function.

The second port can be a USB port. In some embodiments, the object to which the second end of the cable is coupled can be the surgical instrument adapter and the surgical instrument adapter can include a secondary port that is configured to supply power from the secondary power supply to an object that is coupled to the secondary port.

One exemplary method for supplying enhance power during surgery includes supplying power from a first power source to a surgical instrument that is electrically coupled to a generator and supplying power from a second power source through a pass through power tap adapter, with the second power source being electrically isolated from the first power source.

The generator can supply radio frequency and/or ultrasonic energy to the surgical instrument. In some embodiments, supplying power from a second power source through a pass through power tap adapter can include plugging an alternating current adapter of the pass through power tap adapter into an outlet. In other embodiments, the secondary power source can be a battery. The power supplied from the first power source can be used by the surgical instrument to perform a therapeutic function, and the power supplied from the second power source can be used to perform a non-therapeutic function.

In some embodiments, the method can further include performing one or more of the following functions with the surgical instrument electrically coupled to the generator: transecting tissue with an I-blade of the surgical instrument, articulating the surgical instrument when the surgical instrument is a laparoscopic bipolar device, and articulating an end effector of the surgical instrument. Power supplied by the second power source can supply power to sensors associated with the surgical instrument.

This invention will be more fully understood from the following detailed description.

### BRIEF DESCRIPTION OF DRAWINGS

This invention will be more fully understood from the following detailed description taken in conjunction with the accompanying drawings, in which:

FIG. 1 is a schematic illustration of circuitry of traditional systems in the prior art for providing power to surgical devices through a generator;

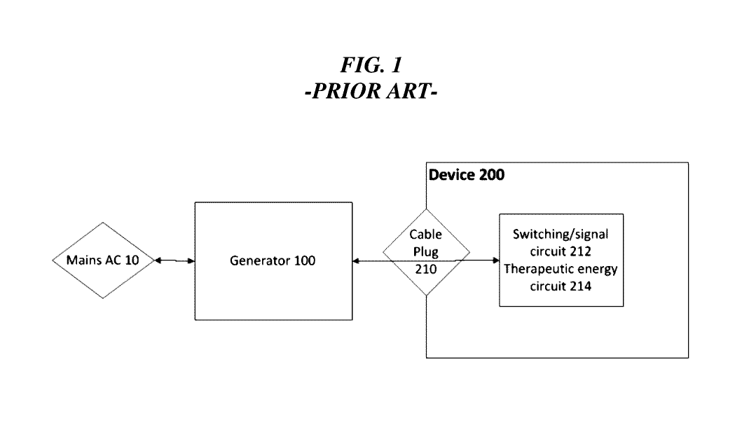

FIG. 2 is a perspective view of one embodiment of a system for providing enhanced power to a surgical device;

FIG. 3 is a close-up view of one portion of the system of FIG. 2;

FIG. 4 is a front perspective view of another embodiment of a system for providing enhanced power to a surgical device;

FIG. 5 is a back perspective view of the system of FIG. 4; and

FIG. 6 is a schematic illustration of one embodiment of circuitry of a system for providing enhanced power to a surgical device.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-numbered components of the embodiments generally have similar features, and thus within a particular embodiment each feature of each like-numbered component is not necessarily fully elaborated upon.

The present disclosure generally relates to devices and methods that provide enhanced power to surgical devices. As explained in detail below, the devices and methods described herein generally provide enhanced power to a surgical device from a secondary power supply operating in parallel to a primary power supply that provides power for therapeutic functions of the device. In some embodiments, the primary power supply provides power to an end effector that directly operates on tissue, while the secondary power supply provides power to ancillary electronics involved in non-therapeutic functionalities of the device such as, by way of non-limiting example, sensors, displays, motors, etc. The primary power supply can be provided by a power supply existing within a generator, such as a battery disposed within the generator that supplies direct current (DC), and/or by a power supply electrically coupled to the generator, such as a wall outlet that supplies alternating current. The secondary power supply can be provided by a secondary power supply typically disposed outside of a generator, such as a battery, power from a wall outlet, or other source of either alternating or direct current, and it can be carried through a pass through power tap adapter, as described in greater detail below.

Subsystems powered by the primary power supply can be wholly isolated from subsystems powered by the secondary power supply, thus helping to prevent faults in one subsystem from affecting the other and providing enhanced power without the need to modify the subsystems powered by the primary power supply. For example, power from both the primary and secondary power supplies can be supplied separately to the device via a connector that is configured to electronically couple to the device, the existing primary power supply (e.g., by way of the generator), and the secondary power supply (e.g., by way of the pass through power adapter). One exemplary connector can include an energy storage device therein for storing energy from the secondary power supply, such that the stored energy can be harvested by the device even if the secondary power supply is currently unavailable. According to any of the systems described herein, additional power can thus be supplied to the device without affecting the structure and/or function of existing subsystems configured to supply primary power.

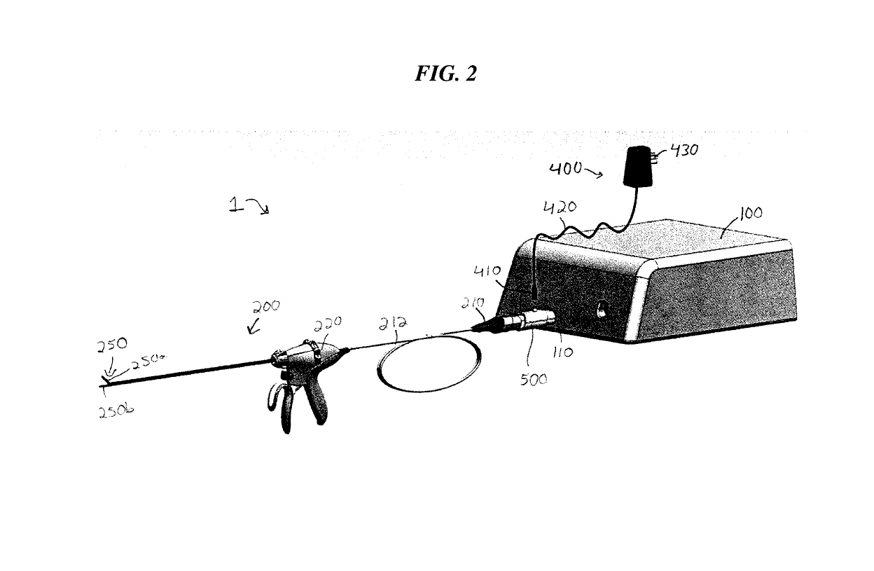

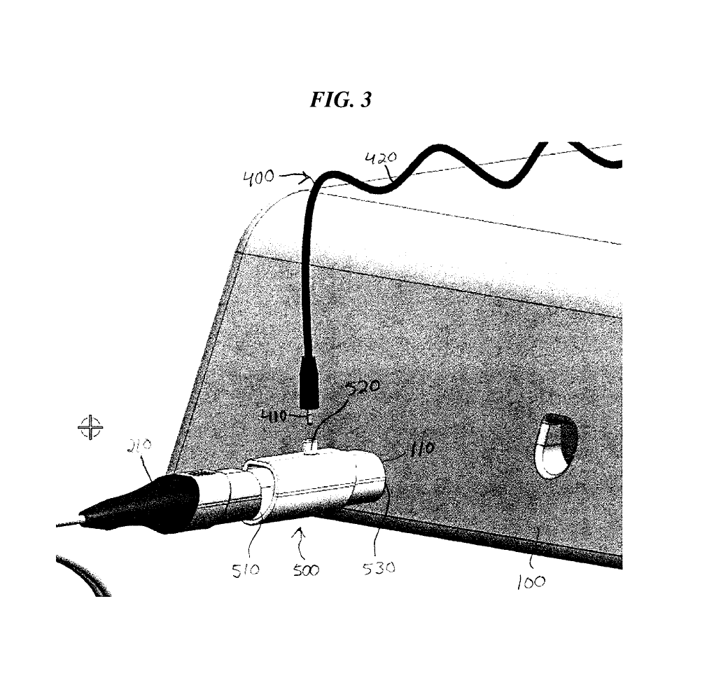

An exemplary system 1 according to the present invention is illustrated in FIGS. 2 and 3. The system 1 includes the generator 100 (housing a primary power supply), a pass through power tap adapter 400 (connected to a secondary power supply), and a surgical device 200 that receives power from both power supplies via a connector 500. In this exemplary embodiment, as with many current systems, the generator 100 is configured to supply energy to a therapeutic portion of the device 200 for operating on tissue. Unlike traditional systems, however, the secondary power supply can be used, by way of the pass through power adapter 400, to provide additional power to the therapeutic portion of the device 200 and/or to provide power to ancillary electronics 216 of the device 200, which, as described in detail below, can relate to non-therapeutic functions of the device 200. Power from both the primary and secondary power supplies can be transmitted to the device 200 via primary and secondary powered subsystems, respectively, which are completely isolated from one another. In this way, the device 200 can receive enhanced power from the secondary power supply without the need for any alterations to the generator 100 and/or the device 200.

The generator 100 can be any generator configured for use with surgical devices. In general, the generator 100 is an existing component of an operating room that is configured for use with a particular surgical device—in this case, with the device 200. However, the generator 100 can be configured for use with any powered surgical device, e.g., any ultrasonic, electrosurgical and/or RF surgical devices. In the illustrated embodiment, the generator 100 can include a housing that contains the primary power supply, the secondary power supply, or both. In some embodiments, the generator 100 can be actuated to control operation of the device 200 in various ways. For example, the generator 100 can provide a drive signal to the device 200 via actuation of an actuation member of the generator 100, e.g., a foot switch. In some embodiments a user can adjust the amount of power supplied to the device 200 via a knob, dial, buttons, or any other actuation feature of the generator 100. In still further embodiments, the generator 100 can be configured to display information about the use of the device 200, such as the amount of power being supplied to the device 200, length of use of the device 200, parameters measured by the device 200 and components associated therewith, etc. More details on generators for use with the systems and methods disclosed herein can be found in U.S. Patent Application Publication No. 2011/0087212, incorporated by reference herein in its entirety.

The device 200 illustrated in FIG. 2 is merely one, non-limiting example of a surgical device configured for use according to the present invention, and it will be appreciated by a person of skill in the art that the device 200 can be any surgical device for which energy is necessary and/or desirable. In the illustrated embodiment, the device 200 is a radiofrequency (RF) device having an end effector 250 that can be used for tissue transection and/or sealing. The device 200 can further include a handle 220 that is in electrical communication with the generator 100 via a cable 212 and the cable plug 210 at a distal end of the cable 212. When activated by the drive signal sent from the generator 100, components of the handle 220 can transmit RF energy to the end effector 250, which can then apply energy to tissue.

The end effector 250 can generally be configured to run on power from the generator 100. However, some components of the device 200 (e.g., components that were released after the purchase of the generator 100) may require additional power from the secondary power supply. For example, in some embodiments, powering the end effector 250 can require additional energy beyond what was required for an end effector of a previously-released product. In still further embodiments, a powered knife component of the device 200 can require additional energy to transect and/or seal tissue, beyond what is required by a traditional end effector 250. One example of such a knife component can include some iterations of an I-blade, which is configured to translate distally and proximally between opposed jaws 250a, 250b and/or to assist in the delivery of energy to the tissue. One exemplary embodiment of an I-blade has a distal portion disposed between opposed tissue-engaging surfaces of the first and second jaws 250a, 250b, with the distal portion including a terminal, vertically disposed cutting edge that extends vertically between the jaws 250a, 250b. In some embodiments, the distal portion of the I-blade can include a first rib extending substantially perpendicular to the terminal, vertically disposed cutting edge and disposed in a track formed in a surface of the first jaw 250a that is opposed to the tissue-engaging surface of the first jaw 250a. Likewise, the distal portion of the I-blade can include a second rib extending substantially perpendicular to the terminal, vertically disposed cutting edge, substantially opposed to the first rib, and disposed in a track formed in a surface of the second jaw 250b that is opposed to the tissue-engaging surface of the second jaw, thus forming an “I-shaped” profile. The exemplary I-blade translates distally and proximally between the jaws 250a, 250b in response to a drive signal. The translation and/or energy delivery functions of the I-blade can, in some instances, require additional power beyond what would typically be supplied by the generator 100, but which can be supplied by the secondary power supply via the adapter 400. More details of exemplary I-blades configured for use with the systems and methods described herein can be found in U.S. Patent Application Publication No. 2012/0116379 and U.S. patent application Ser. No. 14/166,194, entitled “Surgical Devices Having Controlled Tissue Cutting and Sealing” (Docket No. END7329USNP) and filed on Jan. 28, 2014, each of which is incorporated herein by reference in its entirety.

The secondary power supply can further be required to power ancillary electronics 216 of the device 200, which can be any one or more of components for which electrical power is required and/or desirable. In some embodiments, the ancillary electronics 216 can relate to non-therapeutic functions of the device 200. For example, the ancillary electronics 216 can include sensors for sensing a property of the device 200 and/or tissue being acted upon by the device. Ancillary electronics 216 can also include means for conveying information to a user, e.g., lights, a display or displays for providing a clinician with information regarding the surgical device 200, the patient, conditions or properties of the tissue, the generator 100, etc. In some embodiments, the ancillary electronics 216 may comprise a transmission circuit for communicating to other devices the information about the surgical device 200, the patient, conditions or properties of the tissue, the generator 200, etc. Also, in some embodiments, the ancillary electronics 216 may comprise a motor or drive train. The motor and/or drive train may be mechanically coupled to, for example, cause or assist firing of a motor-driven surgical device, articulate an end effector of a surgical device, articulate a laparoscopic bipolar device, etc. In still other embodiments, the ancillary electronics can include an accessory box configured to alter an amount of voltage and/or power supplied to one or more surgical instruments, such as the accessory box disclosed in U.S. Application Serial No. [ ] (Attorney Docket No. 100873-711; END7605USNP), entitled “Surgical Generator Systems and Related Methods,” filed concurrently herewith, the content of which is incorporated by reference herein in its entirety. Other functions and devices or components thereof can be powered by the secondary power supply without departing from the spirit of the present disclosure.

The secondary power supply can be any source of power, e.g., a mains AC power source such as a wall outlet, a battery, a port of the generator 100 and/or of another generator, a transformer, etc. In the illustrated embodiment, the secondary power supply is a wall outlet. While the secondary power supply is configured to provide AC power, it will be appreciated that the secondary power supply can be configured to provide any type of energy to the device 200, which can be either the same or different as the type of power supplied by the primary power supply.

The secondary power supply can be transmitted to the device 200 via any component configured to transmit electrical power from the secondary power supply to the device 200, and, in particular, to the aforementioned components that may require additional power. In the illustrated embodiment, the pass through power adapter 400 transmits power from the secondary power supply (a wall outlet) to the device 200. As shown in FIG. 2, the adapter 400, alternatively referred to as an alternating current adapter when it is configured to be electrically connected to an AC power supply such as current provided through a wall outlet, can have a first plug 410 configured to plug into the connector 500, a second plug 430 configured to plug into the secondary power supply, and a cable 420 extending therebetween.

The connector 500 provides a means for transmitting power from the generator 100 and from the secondary power supply (via the adapter 400) without making any alterations to the generator 100 and/or to the device 200. As illustrated in detail in FIG. 3, the connector 500 has a plug 530 on a proximal end thereof that is configured to plug into a port 110 of the generator 100, which, in some embodiments, is traditionally used to electronically couple the generator 100 directly to the surgical device 200. The connector 500 can further include a port 510 at a distal end thereof that is configured to receive the cable plug 210 of the device 200, which would traditionally plug into the port 110 or previously known connectors lacking the capabilities of the present connector 500. In this way, the device 200 can continue to receive power from the generator 100 as it normally would in the absence of the connector 500. To receive power from the secondary power source (via the adapter 400), the connector 500 can further have a port 520 for receiving the first plug 410 of the adapter 400, thereby electronically coupling the connector 500 and the secondary power supply. Of course, it will be appreciated by a person of skill in the art that the orientation of any of the plugs and ports described herein can be reversed, and/or that the electronic coupling of the connector 500 to the generator 100, device 200, and adapter 400 can be of any type suitable for the necessary power transmission. Furthermore, while the connector 500 of the present embodiment is a separate piece from the generator 100, the device 200, and the adapter 400, in other embodiments, the connector 500 can be integral with either the adapter 400 or the device 200.

In some embodiments, it may be necessary for the device 200 to communicate with the connector 500 regarding the presence and/or necessity of the secondary power supply. For example, the connector 500 can be configured to detect identifying information about a surgical device to which it is coupled. Where the connector 500 detects that the device has components for which the secondary power supply would be desirable and/or necessary, the connector 500 can be configured to send a signal alerting the device 200 and or a user to the availability of the secondary power supply. For example, a light disposed on an actuation portion of the device 200 can light up when the connector 500 indicates that the secondary power supply is present. In some embodiments, the connector 500 can further be configured to detect an amount and/or type of power necessary for use by a particular component that is being actuated by a surgeon. The connector 500 can thus transmit the amount and/or type of power that it detects is necessary for use by the device 200. Additionally or alternatively, the device 200 can be configured to detect the presence of the connector 500, and, when the connector 500 is detected, to send a signal to the connector 500 indicating a need for power from the secondary power supply. The signal can further include information about the amount and/or type of power that is necessary. Generally, the signal can be sent upon actuation by a user of one of the components that requires the secondary power supply. Upon receipt of the signal, the connector 500 can transmit power of a type and in an amount necessary for use by the device 200.

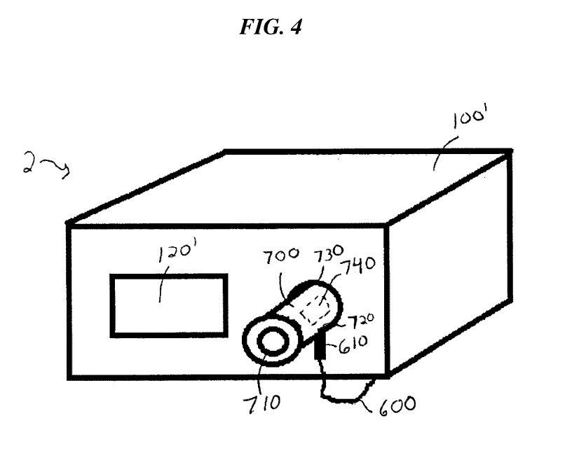

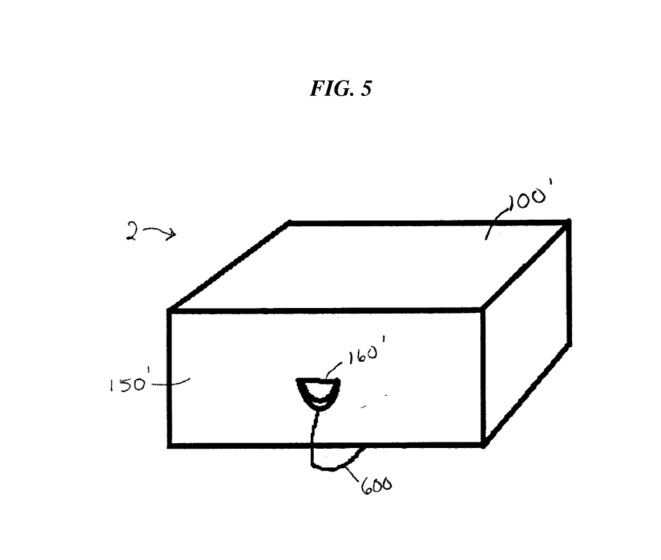

Another exemplary system 2 according to the present disclosure is illustrated in FIGS. 4 and 5. Like the exemplary system 1, the exemplary system 2 includes a generator 100′ housing a primary power supply for powering therapeutic functions of a surgical device (not shown) and a secondary power supply for providing enhanced power for therapeutic functions of the device and/or for powering ancillary electronics of the device. Thus, in this embodiment, both the primary and the secondary power supplies are housed in the generator 100′ itself. In particular, the secondary power supply is a USB port 160′ of the generator 100′, which can supply power to the device via a USB cable 600 electronically coupled to a connector 700, which in turn is electronically coupled to the surgical device. In some embodiments, rather than providing power directly to the device from the USB cable 600, the system 2 can be configured to store power from the USB cable 600 in an energy storage device 740 disposed in the connector 700, as illustrated schematically in FIG. 4 and described in greater detail below.

As shown in FIG. 4, the connector 700 is similar to the connector 500 in that it has a proximal end 730 configured to electronically couple to the generator 100′ and a distal end 710 configured to electronically couple to a surgical device. The connector 700 is further configured to electronically couple to the USB cable 600, in particular via a port 720 that is configured to receive a plug 610 of the USB cable 600.

In some embodiments, as mentioned above, the connector 700 is configured to store power from the USB cable 600 in the energy storage device 740 associated with the connector 700. Once the energy storage device 740 is charged, power from the energy storage device 740 can then be transmitted to the device when needed. Thus, even if there is some failure in the generator 100′, the system 2 can still supply secondary power to the device. The energy storage device 740 can be any component configured to store energy, e.g., a capacitor, a battery, etc. Because charging the energy storage device 740 can be performed any time that the generator 100′ is turned on, the energy storage device 740 can be charged before, during, and after surgical procedures, thereby ensuring a full charge when necessary. Furthermore, once the energy storage device 740 is fully charged, it can be used to supply power for devices used apart from surgery and/or not related to the surgery, e.g., a clinician's mobile phone, by plugging the device into the port 720 of the connector 700. A person skilled in the art will recognize that such an energy storage device can likewise be incorporated within the connector 500 and used for similar purposes without departing from the spirit of the present disclosure.

In some embodiments, the connector 700, the generator 100′, and/or the surgical device coupled to the generator 100′ can be configured to convey information about a charge level of the energy storage device 740. By way of non-limiting example, the connector 700 can send a signal to the generator 100′ indicating a charge status of the energy storage device 740, which can be displayed by the generator 100′ on a display 120′. In this way, a clinician can know when the energy storage device 740 needs to be charged, and/or when the energy storage device 740 can be relied upon for use of enhanced power.

In some embodiments, the connector 700 can have a switch therein to alternately connect the secondary power supply to the energy storage device 740 to charge the device 740 or to connect the secondary power supply directly to the surgical device connected to the generator 100′ via the distal end 710 of the connector 700. For example, the connector 700 can be configured to default to charging the energy storage device 740 and then to transmit power to the surgical device from the energy storage device 740. Upon receipt of a switch signal from the device, the connector 700 can be configured to switch to transmitting power from the secondary power supply directly to the surgical device. It will be appreciated by a person skilled in the art that the connector 700 can be configured to default instead to direct transmission of power to the surgical device from the secondary power supply, and/or that the connector 700 requires some type of switch signal for supplying power in either manner. Furthermore, the surgical device can provide a combined signal instructing the connector 700 to both charge the energy storage device 740 and supply power to the surgical device concurrently.

Notably, when either the energy storage device 740 supplies power to the surgical device or the USB cable 600 supplies power directly from the secondary power supply to the surgical device through the connector 700, the power is in the form of DC power. Because the generator 100′ traditionally supplies AC power, the present embodiment can thus be particularly useful where both types of power are necessary for use by the surgical device. However, it will be appreciated that the secondary power supply can supply AC power as well.

In some embodiments, the USB port 160′ of the generator 100′ may not be readily accessible to a user. In such embodiments, exterior portions of the generator 100′ can be modified to access the USB port 160′ or other secondary energy supply that already exists as part of the generator 100′. For example, in embodiments in which the USB port 160′ is used primarily for installing software updates to the generator 100′, the USB port 160′ may be concealed behind a back cover of the generator 100′. In such instances, the back cover of the generator 100′ may be replaced by a back cover, e.g., the illustrated back cover 150′, having a lumen 152′ formed therein to provide access to the USB port 160′. A change of back covers can be made directly in the field. The back cover 150′ may also include other lumens, e.g., a lumen 154′ to access an electrical port 162′ to plug-in the generator 100′ to a wall outlet. It will be appreciated by a person skilled in the art that any similar modifications may be made to access relevant ports of the generator 100′ without departing from the spirit of the present disclosure, e.g., a side panel or cover of the generator 100′ can be replaced with a panel or cover having additional access ports.

It will be appreciated that the above-described means of providing and/or storing power from a secondary supply is not intended to be limiting. For example, while the energy storage device 740 of the illustrated embodiment is located within the connector 700, it will be appreciated by a person skilled in the art that the energy storage device 740 can be a separate component and/or can be disposed somewhere within the surgical device or other component, such as the previously described accessory box, while not requiring any modification of the existing generator 100′. Similarly, while the secondary power supply is a USB port 160′ on a back panel 150′ of the generator 100′, the secondary power supply can be sourced from any other energy-transmitting port of the generator 100′ or from a separate power source, e.g., a wall outlet.

Throughout all of the above described systems, circuits transmitting power from the primary power supply can be arranged in parallel to circuits transmitting power from the secondary power supply. In some embodiments, the two circuit systems are separated by several layers of insulation. Also, each circuit system can have its own ground. With the two circuit systems arranged in this way, the system effectively operates as two separate devices being powered by two separate power sources. The secondary power supply can therefore supply power to the device without the need for any change to the circuit system transmitting power from the primary source.

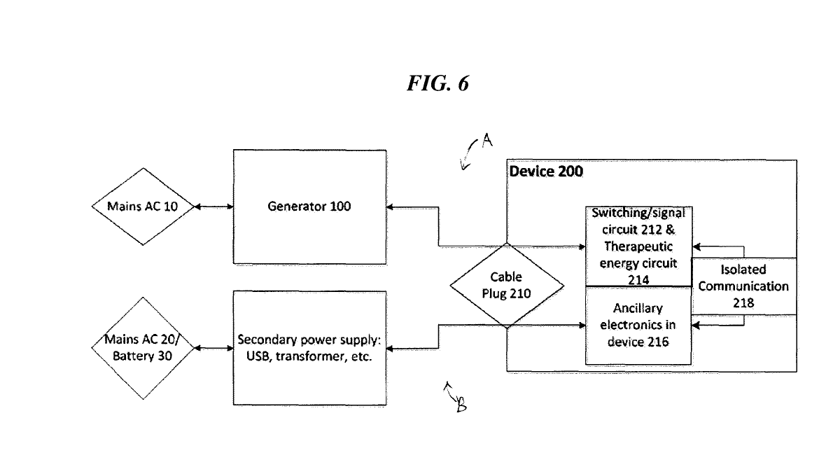

For example, according to the exemplary circuitry of FIG. 6, there is no electrical connection between a power subsystem A powered by the primary power supply and a power subsystem B powered by the secondary power supply. It will be appreciated that the exemplary circuitry of FIG. 6 can be implemented in the systems 1, 2 described herein, or in any other surgical systems in accordance with the present disclosure. The first power subsystem A is powered by the mains AC power source 10 via the generator 100. The generator 100 can transmit power via the cable plug 210 of the device 200 to a switching/signal circuit 212, which can selectively transmit power to a therapeutic energy circuit 214 upon receipt of the drive signal described above. The therapeutic energy circuit 214 can then supply power for therapeutic operations of the device 200 on tissue, e.g., transaction and/or sealing by an end effector of the device. The second power subsystem B can be powered by a second mains AC power source 20 and/or a battery 30 from a secondary power supply, e.g., the USB port 160′, a transformer, and other sources of power described herein or otherwise known to those skilled in the art. This power can be transmitted to a desired location, e.g., the device 200, by way of a component such as an adapter 400 or a cable 600, as described above, in electrical communication with the cable plug 210 of the device 200. Although not illustrated, connectors such as the connectors 500, 700 can be provided as part of the electrical path connecting the device 200 to the primary and secondary power supplies. Power from the secondary power supply can be transmitted in parallel to power from the primary power supply via the cable plug 210 to the ancillary electronics 216 in the device 200. As shown in FIG. 6, the only communication between the first and second circuit systems is isolated communication 218 between the switching/signal circuit 212, the therapeutic energy circuit 214, and the ancillary electronics 216. This isolated communication can be any form of remote communication that does not involve a physical connection, e.g., optical, radio frequency, audible, or inductive. The auxiliary non-therapeutic electronics housed within the instrument may monitor the application of therapeutic energy and reactively power a non-therapeutic function. For example, an inductive current sensor (which can be part of the auxiliary electronics) may detect the application of an AC signal being sent to the instrument end effector for the purpose of tissue treatment. When the application of energy is sensed by the inductive current sensor, the auxiliary electronics determine when to activate and how to control a motor to drive a knife. By way of example, in some instances only a HONEYWELL CSDA series digital/inductive current sensor may be used to monitor the therapeutic circuit. Alternatively, the auxiliary electronics may power a sensor within the device (e.g., temperature sensor to determine tissue temperature) and process that signal to determine a tissue temperature. The auxiliary electronics could then signal to the circuit in communication with the generator the temperature of the tissue. This could be beneficial for enabling a closed loop system in which the generator can determine tissue temperature and adjust power output to drive to a desired temperature. The signal to the generator from the auxiliary electronics my be completed by way of an opto-isolator, by way of example an NTE3040 Optoisolator, available from NTE Electronics, Inc. of Bloomfield, N.J., any radio frequency transmitter and receiver, etc.

The systems disclosed herein can generally be used to provide enhanced power to surgical devices. In one exemplary embodiment, described below, a system can be used to supply secondary power for use of non-therapeutic, ancillary electronics of a surgical device.

In use, a proximal end of a connector can be plugged into a generator in place of a surgical device. The generator can be a traditional generator, already existing in the prior art, that is used to supply power for therapeutic functions of the surgical device when connected thereto. Here, instead, the surgical device can be plugged into a distal end of the connector. A secondary power supply can also be electronically coupled to the connector, either together with or independent of the connection to the generator and/or the surgical device. Where the connector has an energy storage device therein, the secondary power supply can charge the energy storage device.

Once the connector has been electronically coupled to the generator and to the device, a surgeon can activate an actuator of the generator to send a drive signal to the device. Power from the generator is then supplied to an end effector of the device to transect and/or seal tissue. Before, during, or after the operation of the end effector on tissue, a surgeon can activate one or more ancillary electronics of the device, as described herein. Activation of the one or more ancillary electronics can cause the device to send a signal to the connector, which can respond by transmitting power from the secondary power supply to power the one or more ancillary electronics. In some embodiments, the signal can include information about the amount and/or type of power necessary, and the connector can therefore transmit power of the amount and/or type requested. For example, where the connector includes an energy storage device therein, the connector can transmit DC current from the energy storage device to the ancillary electronics upon receipt of a signal indicating that DC current is necessary.

A person skilled in the art will appreciate that the present invention has application in conventional endoscopic and open surgical instrumentation as well application in robotic-assisted surgery.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and its contents are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, hydrogen peroxide gas plasma (i.e. STERRAD), steam.

Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Also, elements or steps from one embodiment can be readily recombined with one or more elements or steps from other embodiments. Further, one skilled in the art will appreciate further features and advantages of the invention based on the above-described embodiments. Accordingly, the invention is not to be limited by what has been particularly shown and described, except as indicated by the appended claims. Finally, all publications and references cited herein are expressly incorporated herein by reference in their entirety.

What is claimed is:

1. A system for supplying enhanced power to a surgical instrument, comprising:
a generator having a first power supply that is configured to supply power to a surgical instrument as part of a first power subsystem; and
a pass through power tap adapter configured to electrically couple to a second power supply as part of a second power subsystem to supply additional power to the surgical instrument that receives power from the generator, the pass through power tap adapter being configured to isolate the second power subsystem from the first power subsystem.

2. The system of claim 1, wherein the power supplied by the first power supply includes alternating current and the power supplied by the second power supply includes direct current.

3. The system of claim 1, wherein the first power subsystem is configured to supply power to a therapeutic end effector of the surgical instrument that receives power from the first and second power subsystems, and the second power subsystem is configured to supply power to a non-therapeutic feature of the surgical instrument that receives power from the first and second power subsystems.

4. The system of claim 1, wherein both the first and second power subsystems are configured to supply power to a therapeutic end effector of the surgical instrument.

5. The system of claim 1, wherein the pass through power tap adapter further comprises:
a connector having a first end configured to couple to the generator, a second end configured to couple to the surgical instrument, and a pass-through outlet; and
an alternating current adapter having a first end configured to electrically couple with the pass-through outlet and a second end configured to electrically couple to the second power supply such that power from the second power supply can be supplied through the alternating current adapter, through the connector, and to the surgical instrument.

6. The system of claim 5, wherein the connector is removably and replaceably coupled to the generator.

7. The system of claim 5, wherein the connector is fixedly coupled to the generator such that the connector cannot readily be decoupled from the generator.

8. The system of claim 1, further comprising a surgical instrument electrically coupled to each of the first and second power subsystems, the surgical instrument being motor-driven.

9. The system of claim 1, further comprising a surgical instrument electrically coupled to each of the first and second power subsystems, the surgical instrument having an actuable I-blade to transect tissue.

10. The system of claim 1, further comprising a means for isolated communication between the first and second subsystems, the means comprising of one of an opto-isolator, inductive current sensor, and RF transmitter and receiver.

11. A system for supplying enhanced power during surgery, comprising:
a generator having a housing with a primary power supply and a secondary power supply disposed therein, the housing including a front panel configured to allow a first port of the generator to be accessible to receive a surgical instrument adapter, the first port providing access to the primary power supply, and a back panel or a side panel configured to allow a second port of the generator to be accessible to provide access to the secondary power supply;
a surgical instrument adapter disposed in the first port such that a surgical instrument can be coupled to the adapter to electrically couple the surgical instrument to the primary power supply; and
a cable coupled at a first end to the second port to electrically couple the cable to the secondary power supply such that power from the secondary power supply can be supplied to an object coupled to a second end of the cable.

12. The system of claim 11, wherein the secondary power supply is configured to provide power to an object coupled to the second end of the cable when power is not being provided to a surgical instrument electrically coupled to the first power supply.

13. The system of claim 11, wherein power provided by the first power supply is configured to power a therapeutic end effector of a surgical instrument and power provided by the second power supply is configured to power a non-therapeutic function.

14. The system of claim 11, wherein the second port is a USB port.

15. The system of claim 11, wherein the object to which the second end of the cable is coupled is the surgical instrument adapter and the surgical instrument adapter further comprises a secondary port configured to supply power from the secondary power supply to an object coupled to the secondary port.

16. A method for supplying enhanced power during surgery, comprising:
supplying power from a first power source to a surgical instrument electrically coupled to a generator; and
supplying power from a second power source through a pass through power tap adapter, the second power source being electrically isolated from the first power source.

17. The method of claim 16, wherein the generator supplies at least one of radio frequency and ultrasonic energy to the surgical instrument.

18. The method of claim 16, wherein supplying power from a second power source through a pass through power tap adapter comprises plugging an alternating current adapter of the pass through power tap adapter into an outlet.

19. The method of claim 16, wherein the power supplied from the first power source is used by the surgical instrument to perform a therapeutic function, and the power supplied from the second power source is used to perform a non-therapeutic function.

20. The method of claim 16, further comprising performing at least one of the following functions with the surgical instrument electrically coupled to the generator: transecting tissue with an I-blade of the surgical instrument, articulating the surgical instrument when the surgical instrument is a laparoscopic bipolar device, and articulating an end effector of the surgical instrument.
